(19) United States
(12) Patent Application Publication
(10) Pub. No.: US 2009/0305413 A1
(43) Pub. Date: Dec. 10, 2009

(54) MULTIPOTENT ADULT STEM CELLS HAVING AN ABILITY OF OCT4 EXPRESSION DERIVED FROM UMBILICAL CORD BLOOD AND METHOD FOR PREPARING THE SAME

(75) Inventor: Kyung Sun Kang, Seoul (KR)

Correspondence Address: MOORE & VAN ALLEN PLLC, P.O. BOX 13706, Research Triangle Park, NC, 27709, US

(73) Assignee: SEOUL NATIONAL UNIVERSITY INDUSTRY FOUNDATION, Seoul (KR)

(21) Appl. No.: 12/095,893

(22) PCT Filed: Nov. 7, 2006
(86) PCT No.: PCT/KR2006/004649

§ 371 Date: Oct. 20, 2008

(30) Foreign Application Priority Data
Dec. 2, 2005 (KR) 10-2005-0117015

(51) Int. Cl. C12N 5/06

(52) U.S. Cl. 435/384; 435/325

(57) ABSTRACT

The present invention relates to multipotent adult stem cells expressing Oct4, derived from umbilical cord blood (UCB) and also these cell are expressing CD29, CD31, CD44, simultaneously, a method for preparing the same, and more specifically to multipotent adult stem cells which are obtained by culturing umbilical cord blood-derived monocytes in a medium containing bFGF (basic fibroblast growth factor) and human serum or plasma. In addition, multipotent adult stem cells expressing Oct-4 from UCB are morphologically spindle or round shaped cells Although the stem cells according to the present invention are adult stem cells, they are multipotent and capable of differentiating into ectodermal-, messodermal-, endodermal-originated tissue or cells including osteogenic cells or nerve cells etc, thus they can be effectively used in the treatment of intractable diseases and incurable diseases.

### TECHNICAL FIELD

The present invention relates to multipotent adult stem cells expressing Oct4 from umbilical cord blood and a method for preparing the same, and more specifically, to multipotent adult stem cells which are obtained by culturing umbilical cord blood-derived monocyte in a medium containing bFGF (basic fibroblast growth factor) and human serum or plasma to isolate.

### BACKGROUND ART

Although many diseases of the past have been cured in our modern society due to the development of life sciences and medical sciences, there still have been inveterate diseases or incurable diseases such as ischemic necrosis, cancer, dementia and a severe burn, and the like. Moreover, there are many problems in the filed of organ transplantation. As a therapy to treat the root cause of these diseases, cell therapy has been in the limelight these days.

Cell therapy is a method for treating or preventing diseases by externally proliferating or sorting autologous stem cells, allogenic stem cells or xenogeneic stem cells, or other methods such as changing biological characteristics of cells. Cell therapy has infinite possibilities in the treatment of inveterate diseases and incurable diseases since it has a very wide range of application areas, for example, proliferating somatic cells collected from the patient himself, other person or other animals or differentiating stem cells into desired cell types to use in the treatment of diseases.

It has been reported that when skeletal myoblasts, endothelial progenitor cells or bone marrow stem cells are transplanted into infracted myocardium, myocardial function is improved (Menasche, P. et al., J. Am. Coll. Cardiol., 41:1078, 2003; Strauer, B. E. et al., Circulation, 106:1913, 2002; and Stamm, C. et al., Lancet, 361:45, 2003). Such improvement of myocardial function is due to formation of mechanical scaffold by arteriogenic cytokine secreted by stem cells or other beneficial cells gathering into ischemic region (Orlic, D. et al., Circ Res, 91:1092, 2002).

Meanwhile, the term “stem cells” refers to cells having not only self-replication ability but also the ability to differentiate into different cell types, and can be divided into totipotent stem cells, pluripotent stem cells, and multipotent stem cells.

Totipotent stem cells are cells having totipotential differentiation properties, which are capable of developing into a complete organism, and the property is possessed by cells up to the 8-cell stage after fertilization of the oocyte by the sperm. When these cells are isolated and transplanted into the uterus, they can develop into a complete organism.

Pluripotent stem cells are cells capable of developing into various cells and tissues derived from the ectodermal, mesodermal and endodermal layers, which are derived from the inner cell mass located inside of blastocysts, generated 4-5 days after fertilization. These cells are called “embryonic stem cells” and can differentiate into various other tissue cells but cannot form new living organisms.

Multipotent stem cells are stem cells differentiating normally into only cell types specific to their tissue and organ of origin, which are involved not only in the growth and development of various tissues and organs during the fetal, neonatal and adult periods but also in the maintenance of adult tissue homeostasis and the function of inducing regeneration upon tissue damage. Tissue-specific multipotent cells are collectively called “adult stem cells”.

Umbilical cord blood-derived adult stem cells known up until now include mesenchymal stem cells capable of differentiating into osteocytes or skeletal muscles (Lee, O. K. et al., Blood, 103:1669, 2004; Gang, E. J. et al., BBRC, 321:102, 2004; Gang, E. J. et al., Stem Cell, 22:617, 2004), heart stem cells capable of differentiating into heart cells (US 2004/0126879) and endothelial progenitor cells (Yamamoto, K. et al., Arterio. Thromb. Vasc. Biol., 24:192, 2004).

Especially, it has been known that lots of adult stem cells are contained in bone marrow, and the adult stem cells differentiate into only bone, cartilage, fat, and so on, because it is derived from mesoderm in embryonic stage. However, it was found that the adult stem cells can also differentiate into nerve cells which are derived from ectoderm, and thus, it is expected to have a very wide range of application areas in the treatment of diseases.

However, because the stem cells are capable of differentiating into a limited variety of cell types, including differentiation into osteocytes or skeletal muscles by mesenchymal stem cells, differentiation into heart cells by heart stem cells, and differentiation into vascular endothelial cells by endothelial progenitor cells, it is difficult to define these cells as true multipotent stem cells.

Accordingly, the present inventors have made an effort to obtain adult stem cells by culturing umbilical cord blood-derived monocyte in a medium containing bFGF and human serum or plasma to isolate, and as a result, found that the adult stem cells have excellent adhesion ability, can express Oct4 which is a specific marker of embryonic stem cells, and differentiate into various tissues such as osteogenic cells and nerve cells, thereby completing the present invention.

### SUMMARY OF THE INVENTION

It is an object of the present invention to provide a method for isolating multipotent adult stem cells having the ability to express Oct4, which is derived from umbilical cord blood using a medium containing bFGF and human serum or plasma.

It is another object of the present invention to provide a method for preparing adult stem cells, the method comprising the steps of culturing umbilical cord blood-derived monocyte in a medium containing bFGF and human serum or plasma, and recovering the adult stem cells, wherein the adult stem cells show the following characteristics:
(a) having the ability to express Oct4;
(b) showing positive immunological responses to all of CD24, CD29, CD31, CD 44, CD33, CD45 and CD49B, and negative immunological responses to CD34, CD51/61, CD62L, CD62P, CD90, CD133 and CD135;
(c) growing on plastics and showing round-shaped or spindle-shaped morphological features; and
(d) having the ability to differentiate into the cells derived from mesoderm, endoderm and ectoderm.

In the present invention, the content of bFGF and human serum or plasma is preferably 1˜30 ng/ml and 5˜30%, respectively, and the human serum or plasma is autologous or allologous, and the medium is α-MEM.

The present invention also provides adult stem cells obtained by the above described method and showing the following characteristics:
(a) having the ability to express Oct4;
(b) showing positive immunological responses to all of CD24, CD29, CD31, CD33, CD45 and CD49B, and negative immunological responses to CD34, CD51/61, CD62L, CD62P, CD90, CD133 and CD135;
(c) growing on plastics and showing round-shaped or spindle-shaped morphological features; and
(d) having the ability to differentiate into the cells derived from mesoderm, endoderm and ectoderm.

In the present invention, the adult stem cells preferably additionally show positive immunological responses to one or more selected from the group consisting of SH-2, SH-3 and SH-4, and preferably show positive or negative immunological responses to CD44, CD105 and CD117. Moreover, the cells derived from mesoderm are preferably osteogenic cells or endothelial cells, and the cells derived from ectoderm are preferably nerve cells.

Another features and embodiments of the present invention will be more clarified from the following “detailed description” and the appended “claims”.

### BRIEF DESCRIPTION OF DRAWINGS

FIG. 1 is a photograph of umbilical cord blood-derived multipotent adult stem cells according to the present invention.

FIG. 2 shows the immunological characteristics of umbilical cord blood-derived multipotent stem cells, measured by flow cytometry. A and E: control groups; B: CD34; C: CD45; D: SH-2; and F: SH-3.

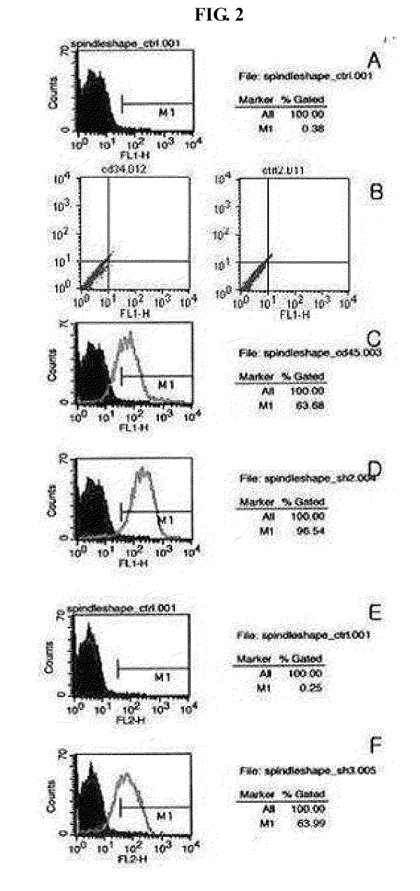

FIG. 3 shows the results of PAS staining conducted to examine the expression or non-expression of antigens in multipotent adult stem cells according to the present invention.

FIG. 4 is photographs of the multipotent adult stem cells derived from umbilical cord blood, differentiated into osteogenic cells (A and B), and the results of Von-Kossa staining (C and D).

FIG. 5 shows binding between umbilical cord blood-derived multipotent adult stem cells and specific antigens. A and B illustrate that the multipotent stem cells show positive responses to NSE (neuron-specific enolase), a neuron-specific antigen, and GFAP (glial fibrillary acidic protein), an astrocyte-specific antigen, respectively, and C and D show control groups.

FIG. 6 shows Oct4 expression in umbilical cord blood-derived multipotent stem cells after immunostained [A: phase contrast; B: Oct4; C: merge].

FIG. 7 shows the expression of SH-4 and Oct4 in umbilical cord blood-derived multipotent stem cells after immunostained [A: SH-4; B: Oct4; C: merge].

### DETAILED DESCRIPTION OF THE INVENTION, AND PREFERRED EMBODIMENTS

The present invention relates to a method for isolating multipotent adult stem cells expressing Oct4 from umbilical cord blood, the isolation method is as follows.

At first, 70˜100 ml of umbilical cord blood sample was diluted in PBS at a ratio of 1:1 to stir, and separated on ficoll at a Ficoll pague-umbilical cord blood ratio of 15:25. For this purpose, on 15 ml of a ficoll solution, the above blood sample solution (diluted in PBS at a ratio of 1:1) was centrifuged to obtain a white layer (monocytic layer), and the white layer was centrifuged. Herein, upper layer was completely removed and precipitation was stored in ice.

Next, the precipitation was shaken smoothly to disperse, and added with 10 ml of culture broth to disperse uniformly, and then centrifuged at 1200 rpm for 5 min, from which upper layer is completely removed and added with 10 ml of culture broth to centrifuge at 1000 rpm for 5 min (this process is repeated two times), thus obtaining cells.

α-MEM (Gibco) containing 5˜30% of human serum or plasma and 10 ng/ml of bFGF (basic fibroblast growth factor)[Roche, Seitzerland] was added to the obtained cells, and optimum number of cells (about 3˜5×10⁶/ml) were plated in a culture vessel to culture for 3 days. After 3 days, relatively heavy cells such as RBC (red blood cell) exist in the lower layer of culture broth, and adult stem cells to be isolated exist in the upper layer. The upper layer is carefully collected to move into a new culture vessel to culture, thus finally obtaining adult stem cells having the ability to express Oct4.

Methods for obtaining multipotent stem cells expressing desired surface antigens from the obtained stem cell broth include FACS method using flow cytometer with sorting function (Int. Immunol., 10(3):275, 1998), a method using magnetic beads, and panning method using an antibody specifically recognizing multipotent stem cells (J. Immunol., 141(8):2797, 1998). Also, methods for obtaining multipotent stem cells from a large amount of culture broth include a method where antibodies, which is expressed on the surface of cells to specifically recognize molecules (hereinafter, referred to as “surface antigens”), are used alone or in combination as columns.

Flow cytometric sorting methods may include droplet charge method and cell capture method. In any of these methods, an antibody specifically recognizing an antigen on the cell surface is fluorescently labeled, the intensity of fluorescence emitted from the antibody bonded with the molecule expressed on the surface of the cell is converted to an electric signal whereby the amount of antigen expressed on cells can be quantified. It is also possible to separate cells expressing a plurality of surface antigens by combination of fluorescence types used therefore. Examples of fluorescent labels which can be used in this case include FITC (fluorescein isothiocyanate), PE (phycoerythrin), APC (allo-phycocyanin), TR (Texas Red), Cy 3, CyChrome, Red 613, Red 670, TRI-Color, Quantum Red, etc.

FACS methods using flow cytometer include: a method where the obtained stem cell broth is collected, from which cells are isolated by, for example, centrifugation, and stained directly with antibodies; and a method where the cells are cultured and grown in a suitable medium and then stained with antibodies. The staining of cells is performed by mixing a primary antibody recognizing a surface antigen with a target cell sample and incubating the mixture on ice for 30 minutes to 1 hour. When the primary antibody is fluorescently labeled, the cells are isolated with a flow cytometer after washing. When the primary antibody is not fluorescently labeled, cells reacted with the primary antibody and a fluorescently-labeled secondary antibody having binding activity to the primary antibody are mixed after washing, and incubated on ice water for 30 minutes to 1 hour. After washing, the cells stained with the primary and secondary antibodies are isolated with a flow cytometer.

The method using magnetic beads allows mass isolation of cells expressing the target surface antigens. Although cell purity by this isolation method is lower than that by the above-described method using the flow cytometer, it can secure sufficient cell purity by repeated purification.

As a marker, there may be an embryonic stem cell-specific protein, hematopoietic antigens, surface antigens of mesenchymal cells, and neuron-specific antigens of the nervous system, and the like. The embryonic stem cell-specific proteins include Oct4, and the like and the hematopoietic antigens include CD34 and CD45, and the like and the surface antigens of mesenchymal cells include SH-2 and SH-3, and the like and the neuron-specific antigens of the nervous system include NSE and GFAP, and the like. The single or combined use of antibodies recognizing the above-described surface antigens enables desired cells to be obtained.

The inventive multipotent adult stem cells show positive response to monocyte-macrophage antigen CD45, negative response to hematopoietic lineage antigen CD34 and have the ability to express Oct4, thus it is assumed to be stem cells in which differentiation into monocytes from hematopoietic cells was proceeding.

### EXAMPLES

Hereinafter, the present invention will be described in more detail by examples. However, it is obvious to a person skilled in the art that these examples are for illustrative purpose only and are not construed to limit the scope of the present invention.

### Example 1

### Isolation of Adult Stem Cells from Umbilical Cord Blood

Umbilical cord blood was collected from full-term and preterm newborns in Seoul National University Hospital and Samsung Cheil Hospital according to Institutional Review Board guidelines.

70-100 ml of the collected umbilical cord blood sample was diluted in PBS at a ratio of 1:1 to stir. Then, the blood sample was separated on ficoll at a ratio of 15:25, in which the blood sample (diluted in PBS at a ratio of 1:1) was spilled smoothly onto 15 ml of Ficoll solution to cause layer separation, followed by centrifugation at 2500 rpm for 20 minutes. After the centrifugation, three different layers were formed starting from the bottom, among them, buffy coat (the middle layer:monocytic layer) was taken with a micropipette, and washed three times with HBSS, followed by centrifugation at 1800 rpm for 15 minutes, from which upper layer is completely removed and a precipitation was stored in ice.

The precipitation was shaken smoothly to disperse, and added with 10 ml of culture broth to disperse uniformly, and then centrifuged at 1200 rpm for 5 min, from which upper was completely removed and added with 10 ml of culture broth to centrifuge at 1000 rpm for 5 min (this process is repeated two times), thus obtaining cells.

α-MEM (Gibco) containing 5˜30% of human serum or plasma and 10 ng/ml of bFGF (basic fibroblast growth factor)[Roche, Seitzerland] was added to the obtained cells, and optimum number of cells (about 3˜5×10⁶/ml) were plated in a culture vessel to culture for 3 days. After 3 days, relatively heavy cells such as RBC (red blood cell) existed in the lower layer of culture broth and adult stem cells to be isolated existed in the upper layer, from which the upper layer is carefully separated to move into a new culture vessel to culture, thus finally obtaining adult stem cells.

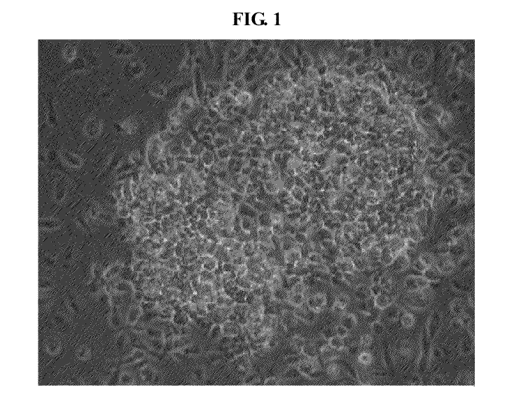

FIG. 1 is microphotograph of multipotent adult stem cells derived from umbilical cord blood according to the present invention.

### Example 2

### Immunological Characteristics of Umbilical Cord Blood-Derived Multipotent Adult Stem Cells

To examine the immunological characteristics of the umbilical cord blood-derived multipotent adult stem cells obtained in Example 1, the pattern of cell surface antigen expression was analyzed. For this purpose, 2×10⁶-10⁷ of the cells cultured in Example 1 were washed with PBS solution and incubated with their corresponding antibodies at room temperature. The expression or non-expression of the antigens was analyzed with a flow cytometer. Also, PAS staining periodic acid Schiff staining) was conducted.

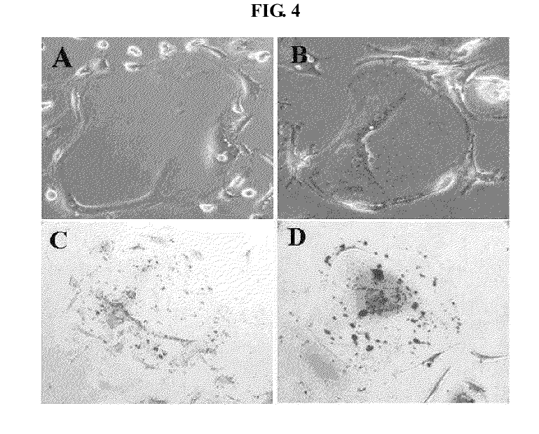

As a result, as shown in FIG. 4, the inventive umbilical cord blood-derived multipotent adult stem cells showed 63.38%, 96.54% and 63.99% of positive responses to CD45, SH-2 and SH-3, respectively, and more than 90% of negative response to CD34. Also, immunophenotypes of other antigens were analyzed and as a result, the immunological characteristics of the multipotent adult stem cells were all negative for CD51/61, CD62L, CD62P, CD133, CD135, CD90, positive for CD29, positive for CD44, positive for CD49B, positive or negative for CD105(SH-2), and negative for CD90.

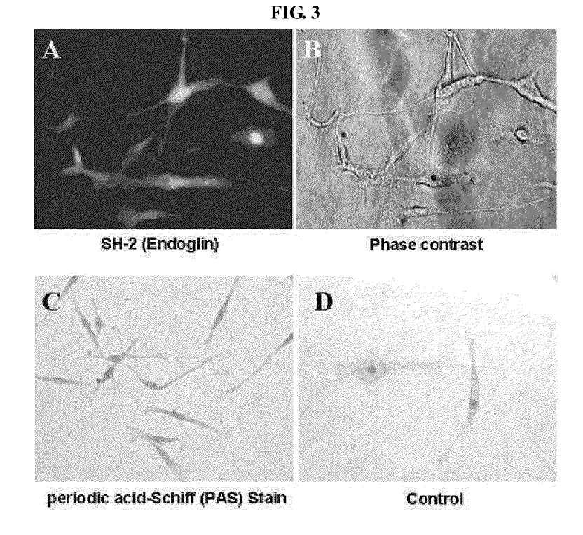

Meanwhile, as shown in FIG. 3, the multipotent adult stem cells showed positive response in PAS staining.

### Example 3

### Differentiation of Umbilical Cord Blood-Derived Multipotent Adult Stem Cells into Osteogenic Cells

The umbilical cord blood-derived multipotent adult stem cell broth obtained in Example 1 was diluted in 1 ml of osteogenesis-inducing medium (0.1 μmol/L dexamethasone (Sigma, USA), 0.05 mmol/L ascorbic acid-2-phosphate (Sigma, USA), 10 mmol/L beta-glycophosphate (Sigma), and 5-30% human serum or plasma) to count the number of cells. Then, the cells were cultured in a flask (5% CO₂; 37° C.; medium replaced one time at 3-4-day intervals) to induce the differentiation of the multipotent adult stem cells into osteogenic cells. 14 days after the initiation of the culture, it was confirmed by Von-Kassa staining that the umbilical cord blood-derived multipotent adult stem cells differentiated into osteogenic cells (see FIG. 4).

### Example 4

### Differentiation of Umbilical Cord Blood-Derived Multipotent Adult Stem Cells into Nerve Cells

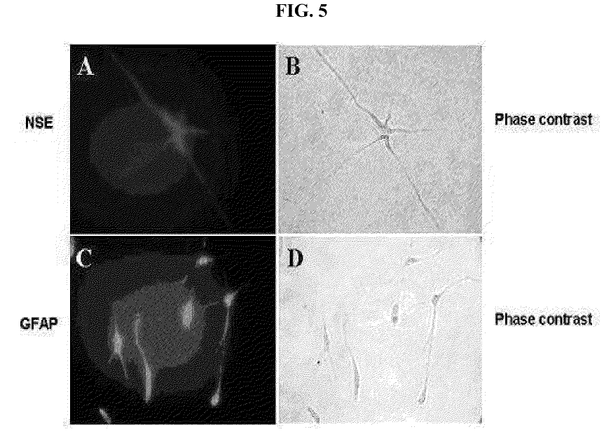

The umbilical cord blood-derived multipotent adult stem cell broth obtained in Example 1 were diluted in 1 ml of neural medium (containing 10 ng/ml basic fibroblast growth factor (Roche, Switzerland), 10 ng/ml human epidermal growth factor (Roche, Switzerland), 10 ng/ml human neural growth factor (Invitrogen, USA) and 5-30% human serum or plasma). The cells were cultured on a flask in 5% CO₂ incubator at 37° C. to induce differentiation of the multipotent adult stem cells into nerve cells. 14 days after the initiation of the culture, it was confirmed that the multipotent stem cells showed positive responses to NSE (neuron-specific enolase), a neuron-specific antigen, and GFAP (glial fibrillary acidic protein), an astrocyte-specific antigen. This suggests that the umbilical cord blood-derived multipotent adult stem cells differentiated into nerve cells (see FIG. 5).

### Example 5

### Oct4 Expression of Umbilical Cord Blood-Derived Multipotent Adult Stem Cells

The adult stem cells obtained in Example 1 were washed with PBS three times, added with 4% of paraformaldehyde solution and allowed to react for 5˜10 minutes at room temperature to fix the cells, followed by washing with PBS three times to remove the paraformaldehyde solution. The cells fixed by the above process were added with 0.3% of triton X-100 (in PBS) and allowed to react for 5 minutes at room temperature to make a condition where an exterior material is permeable into cells, followed by washing with PBS three times to remove the triton X-100.

The resulting cells were added with 10% of NGS, allowed to react for 30 minutes at room temperature, and added with PBS containing primary antibody (diluted at a ratio of 1:200) and NGS (diluted at a ratio of 1:100), and then allowed to react at 4° C. overnight, followed by washing with PBS three times to remove the reaction solution. After adding with PBS containing secondary antibody (diluted at a ratio of 1:200) and NGS (diluted at a ratio of 1:100), the resulting cells were allowed to react at 37° C. for 1 hour to wash with PBS three times to remove the reaction solution, followed by mounting to observe with a microscope.

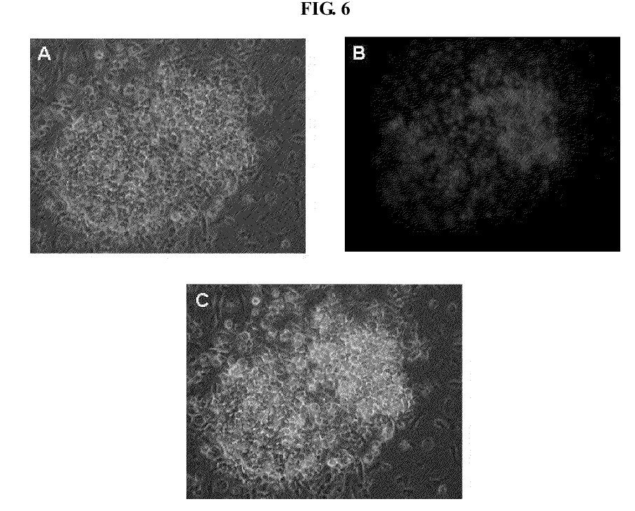

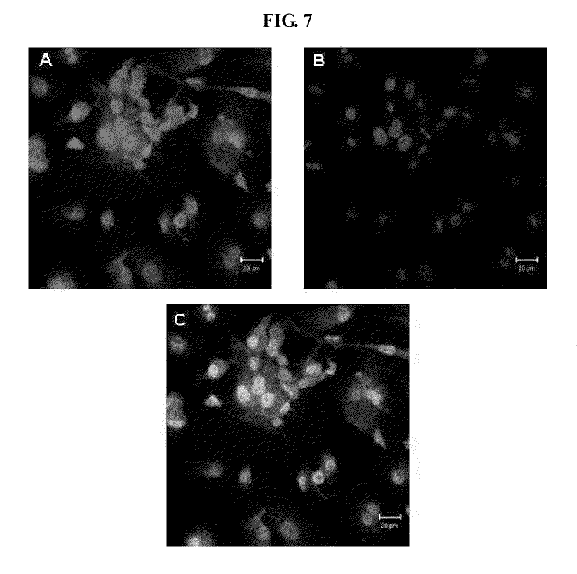

As a result, as shown in FIG. 6, the adult stem cells according to the present invention showed positive response to Oct4 which is a protein expressing embryonic stem cells, as well as a cell marker of undifferentiation. Furthermore, as shown in FIG. 7, it is conformed that the expression of Oct4 and SH-4 was showed simultaneously in the adult stem cells according to the present invention.

Oct4 which is expressed in the adult stem cells according to the present invention is a transcription factor expressed in embryonic stem cells, which is involved in preventing cell differentiation, disappears after natural cell differentiation begins, and is known as a marker of pluripotent stem cells (Donovan, P. J., Nature Genet., 29:246, 2001; Pesce, M. and Scholer, H. R., Stem Cells, 19:271, 2001). Therefore, the result that the adult stem cells according to the present invention express the Oct4 which is a marker of pluripotent embryonic stem cells, clearly proves the fact that the adult stem cells according to the present invention is pluripotent.

### INDUSTRIAL APPLICABILITY

The present invention provides a method for isolating multipotent adult stem cells expressing Oct4, derived from umbilical cord blood using a medium containing bFGF and human serum or plasma. Although the stem cells according to the present invention are adult stem cells, they are multipotent and capable of differentiating into osteogenic cells or nerve cells etc, thus they can be effectively used in the treatment of intractable disease and incurable disease.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for preparing adult stem cells, the method comprising the steps of culturing umbilical cord blood-derived monocytes in a medium containing bFGF and human serum or plasma, and recovering the adult stem cells, wherein the adult stem cells show the following characteristics:
(a) having the ability to express Oct4;
(b) showing positive immunological responses to all of CD24, CD29, CD31, CD33, CD45 and CD49B, and negative immunological responses to CD34, CD51/61, CD62L, CD62P, CD90, CD133 and CD135;
(c) growing on plastics and showing round-shaped or spindle-shaped morphological features; and
(d) having the ability to differentiate into the cells derived from mesoderm, endoderm and ectoderm.

2. The method for preparing adult stem cells according to claim 1, wherein the content of the bFGF and human serum or plasma is 1˜30 ng/mL and 5˜30%, respectively.

3. The method for preparing adult stem cells according to claims 1, wherein the human serum or plasma is autologous or allologous.

4. The adult stem cells according to claim 1, wherein the medium is α-MEM.

5. Adult stem cells obtained by the method of claim 1, which show the following characteristics:
(a) having the ability to express Oct4;
(b) showing positive immunological responses to all of CD24, CD29, CD31, CD33, CD45 and CD49B, and negative immunological responses to CD34, CD51/61, CD62L, CD62P, CD90, CD133 and CD135;
(c) growing on to plastics and showing round-shaped or spindle-shaped morphological features; and
(d) having the ability to differentiate into the cells derived from mesoderm, endoderm and ectoderm.

6. The adult stem cells according to claim 5, wherein the adult stem cells additionally show positive immunological responses to one more selected from the group consisting of SH-2, SH-3 and SH-4.

7. The adult stem cells according to claim 5, wherein the adult stem cells show positive or negative immunological responses to CD44, CD105 and CD117.

8. The adult stem cells according to claim 5, wherein the cells derived from mesoderm are osteogenic cells or endothelial cells.

9. The adult stem cells according to claim 5, wherein the cells derived from ectoderm are nerve cells.

10. Adult stem cells obtained by the method of claim 4, which show the following characteristics:
(a) having the ability to express Oct4;
(b) showing positive immunological responses to all of CD24, CD29, CD31, CD33, CD45 and CD49B, and negative immunological responses to CD34, CD51/61, CD62L, CD62P, CD90, CD133 and CD135;
(c) growing on to plastics and showing round-shaped or spindle-shaped morphological features; and
(d) having the ability to differentiate into the cells derived from mesoderm, endoderm and ectoderm.
